(19) United States
(12) Patent Application Publication
(10) Pub. No.: US 2016/0317426 A1
(43) Pub. Date: Nov. 3, 2016

(54) COMPOSITION COMPRISING A SILICONE ELASTOMER, A SILICONE OIL, A PASTY FATTY SUBSTANCE AND AN APOLAR HYDROCARBON-BASED COMPOUND, AND USE AS AN ANTIMIGRATION COMPOSITION

(71) Applicant: L'OREAL, Paris (FR)

(72) Inventors: Virginie PEREZ-NOWAK, Cachan (FR); Annabelle SERVAIS, Vincennes (FR)

(73) Assignee: L'OREAL, Paris (FR)

(21) Appl. No.: 15/103,686

(22) PCT Filed: Dec. 22, 2014
(86) PCT No.: PCT/EP14/79072

§ 371 Date: Jun. 10, 2016

(30) Foreign Application Priority Data
Dec. 23, 2013 (FR) 1363423

(51) Int. Cl. A61K 8/891; A61Q 1/04; A61K 8/97; A61Q 19/00; A61K 8/92; A61K 8/58; A61K 8/31; A61K 8/25; A61K 8/81

(52) U.S. Cl. CPC A61K 8/891; A61K 8/25; A61Q 1/04; A61K 8/97; A61K 8/8194; A61K 8/92; A61K 8/585; A61K 8/31; A61Q 19/001; A61K 2800/43; A61K 2800/594; A61K 2800/5922

(57) ABSTRACT

The present invention relates to a cosmetic composition, preferably for making up and/or caring for the lips, comprising: at least 6% by weight, expressed as solids, relative to the weight of the composition, of at least one organopolysiloxane elastomer conveyed in a first oil; at least one volatile or non-volatile non-phenyl silicone oil; at least one apolar hydrocarbon-based compound, which is liquid or solid at room temperature; at least one pasty fatty substance. The invention also relates to a process for making up and/or caring for the lips, comprising the application of the said composition to the lips; this composition being applied alone or before a pigmented or unpigmented composition. The invention also relates to the use of the composition for giving the lips a smooth appearance.

The present invention relates to a solid to creamy cosmetic composition intended more particularly for making up and/or caring for the lips, comprising a given content of silicone elastomer conveyed in a first oil, at least one non-phenyl silicone oil, at least one hydrocarbon-based apolar compound, which is liquid or solid at room temperature, and at least one pasty fatty substance.

The invention also relates to a process for making up and/or caring for the lips, which consists in applying the said composition, and also to the use of this composition for preventing or limiting the migration of the deposit resulting from the application to the lips of a pigmented and/or glossy composition, or alternatively for giving the lips a smooth appearance.

The development of fluid or solid compositions dedicated to making up and/or caring for the lips, which are stable and endowed with satisfactory properties in terms of application (glidance on application, ease of spreading and fineness of the deposit), but also in terms of the makeup effect of the deposit on the lips, for instance the coverage and the absence of migration of the deposit, preferably without becoming tacky, is an ongoing objective.

Until very recently, migration of the deposits obtained from liquid or solid lipstick around the lips was avoided by using a coloured pencil to define the contour thereof. The use of products of this type is often problematic since the exact same shade needs to be found for the pencil and for the lipstick, so as to obtain an attractive makeup result.

Recently, a few uncoloured pencils have appeared on the market and thus solve the colour problem. It nevertheless remains important to clearly define the contour of the lips, which requires a certain dexterity. Furthermore, the lead of these pencils is often hard and relatively unpleasant to use.

The invention proposes a lip composition in pigmented or unpigmented wand or cream form, which makes it possible to totally or appreciably reduce the migration of lip makeup compositions, whether they are liquid or solid, and the makeup result of which is virtually invisible and does not impair either the makeup performance qualities of the abovementioned lipstick compositions.

Thus, according to one of its aspects, a subject of the present invention is a cosmetic composition, which is advantageously solid or creamy, preferably for making up and/or caring for the lips, comprising
at least 6% by weight, expressed as elastomer solids, relative to the weight of the composition, of at least one organopolysiloxane elastomer conveyed in a first oil,
at least one volatile or non-volatile non-phenyl silicone oil,
at least one apolar hydrocarbon-based compound, which is liquid or solid at room temperature,
at least one pasty fatty substance.

A subject of the invention is also a process for making up and/or caring for the lips, in which the said composition is applied.

The composition may be applied alone or as a first coat before the application of a second pigmented or unpigmented composition, more particularly a makeup composition, which is liquid (fluid, gloss) or solid (lipstick).

Thus, the composition according to the invention is applied, preferably to bare lips, the composition is optionally left to dry, and then another pigmented or unpigmented, liquid or solid composition is applied.

The present invention similarly relates to the use of such a composition for preventing or limiting the migration of the deposit resulting from the application to the lips of another pigmented or unpigmented, liquid or solid composition.

Finally, a subject of the invention is the use of the composition according to the invention to give the lips a smooth appearance.

It has in fact been found, entirely surprisingly, that such a composition, applied beforehand to a pigmented and/or glossy makeup composition, makes it possible to limit considerably the migration of the deposit of this makeup composition into the fine lines around the lips.

The existence of such a result without substantially modifying the appearance of the deposit of the composition applied thereafter, or its gloss or persistence properties, or even impairing its properties during the very application of this second makeup composition, was found.

The presentation form (stick or cream) of the invention furthermore allows easier use than a pencil, since it does not require any particular dexterity.

In addition, irrespective of the presentation form in which it is found, the composition according to the invention is easy to apply, which is especially reflected in terms of glidance. Moreover, in the case of solid forms, the composition according to the invention is both easy to disintegrate and solid enough not to crack during application.

Further, when it is in the form of a cream, the composition spreads easily in the form of a uniform film, without lumps.

Moreover, irrespective of the form of the composition (solid or cream) it is found that the lips have a considerably smoother appearance once it has been applied. Furthermore, since the deposit formed by the composition is virtually imperceptible to the naked eye and is not glossy, it gives the illusion of bare lips

However, other characteristics and advantages of the invention will emerge more clearly on reading the description and the examples that follow.

The composition according to the invention is homogeneous and stable at room temperature. The term “stable” composition especially means that it does not show any exudation or phase separation, in particular after 1 month or even 2 months at 47° C.

According to a first particular embodiment, the composition is in solid form at 20° C.

The term “solid” cosmetic composition means the form of the composition at 20° C., and in particular the term “solid” means a composition whose hardness at 20° C. and at atmospheric pressure (760 mmHg) is greater than or equal to 30 Nm⁻¹ when it is measured according to the protocol described below.

In a particularly preferred manner, the composition according to the invention is a makeup composition, preferably for the lips, for example a solid lipstick, which may be, for example, in the form of a stick or cast in a jar or a dish.

According to a second embodiment, the composition is in the form of a fluid texture at 20° C.

The term “fluid” means a creamy or even pasty texture, which in particular means a composition that is not solid at room temperature (20° C.) and for which it is possible to evaluate the characteristics via penetrometry measurements.

The hardness of the solid composition is measured according to the following protocol:

Protocol for Measuring the Hardness

The lipstick is stored at 20° C. for 24 hours before measuring the hardness.

The hardness may be measured at 20° C. via the “cheese wire” method, which consists in transversely cutting a wand of product, which is preferably a circular cylinder, by means of a rigid tungsten wire 250 μm in diameter, by moving the wire relative to the stick at a speed of 100 mm/minute.

The hardness of the samples of compositions of the invention, expressed in Nm⁻¹, is measured using a DFGS2 tensile testing machine from the company Indelco-Chatillon.

The measurement is repeated three times and then averaged. The average of the three values read using the tensile testing machine mentioned above, noted Y, is given in grams. This average is converted into newtons and then divided by L which represents the longest distance through which the wire passes. In the case of a cylindrical wand, L is equal to the diameter (in metres).

The hardness is converted into Nm⁻¹ by the equation below:

(Y×10⁻³×9.8)/L

For a measurement at a different temperature, the stick is stored for 24 hours at this new temperature before the measurement.

According to this measuring method, the composition according to the invention advantageously has a hardness at 20° C. and at atmospheric pressure of greater than or equal to 30 Nm⁻¹. According to one particular mode, the hardness at 20° C. and at atmospheric pressure is greater than or equal to 50 Nm⁻¹.

Preferably, the composition according to the invention has a hardness at 20° C. of less than 500 Nm⁻¹, especially less than 400 Nm⁻¹ and preferably less than

300 Nm⁻¹. Preferably, the composition has a hardness of between 50 and 110 Nm⁻¹.

The texture of the cream composition is evaluated via its penetrometry measurement.

Protocol for Measuring the Penetrometry:

This measurement consists in determining the resistance force opposing the composition during its penetration, under specific conditions.

In our case, the penetrometry is measured using a texturometer sold under the name TA-TX2i by the company Rheo with a 5 kg force sensor equipped with a P/4 cylindrical spindle 4 mm in diameter.

The samples are prepared by pouring the composition at the end of manufacture into a metal cylinder made of brass, which is open on both sides (inside diameter: 25.4 mm; height: 31.8 mm) placed on a metal plate made of brass.

The surface of the product is levelled off so as to have a flat, smooth and uniform surface. Three measurements are taken at 20° C.±1° C. on the same container 24 hours after pouring onto the face of the composition. The texturometer travels at a speed of 0.5 mm/s and penetrates into the sample to a depth of 5 mm.

Preferably, the composition has at 20° C. a penetrometry of between 50 g and 200 g and preferably between 70 g and 150 g.

The terms “between” and “ranging from” should be understood as including the limits.

The composition according to the invention is cosmetic and advantageously comprises a physiologically acceptable medium, i.e. a medium that is particularly suitable for applying a composition of the invention to the lips.

The physiologically acceptable medium is generally adapted to the nature of the support onto which the composition has to be applied, and also to the appearance under which the composition has to be packaged.

Preferably, the composition comprises less than 2% by weight and more advantageously less than 0.5% by weight of water. Particularly preferably, the composition according to the invention is anhydrous. The term “anhydrous” especially means that water is preferably not deliberately added to the compositions, but may be present in trace amounts in the various compounds used in the compositions.

Silicone Elastomer

The composition according to the invention comprises at least one organopolysiloxane elastomer (also referred to as silicone elastomer) conveyed in a first oil, in particular in the form of an organopolysiloxane elastomer gel.

Preferably, the said oil is a silicone oil and/or a hydrocarbon-based oil, which is volatile or non-volatile, preferably a silicone oil, which will also be described hereinbelow.

For example, the said composition comprises at least one organopolysiloxane elastomer conveyed in at least one silicone oil, which is preferably non-volatile, especially having the INCI name Dimethicone.

The term “organopolysiloxane elastomeK or “silicone elastomer” means a soft, deformable organopolysiloxane with viscoelastic properties and especially with the consistency of a sponge or soft sphere. Its modulus of elasticity is such that this material withstands deformation and has a limited capacity for extension and contraction. This material is capable of regaining its original shape after stretching.

It is more particularly a crosslinked silicone elastomer.

The silicone elastomer particles are conveyed in the form of a gel formed from an elastomeric organopolysiloxane included in at least one hydrocarbon oil and/or one silicone oil.

In these gels, the organopolysiloxane particles are often non-spherical particles.

Non-Emulsifying Organopolysiloxane Elastomer

Thus, the organopolysiloxane elastomer may be obtained by crosslinking addition reaction of diorganopolysiloxane containing at least one hydrogen bonded to silicon and of diorganopolysiloxane containing ethylenically unsaturated groups bonded to silicon, especially in the presence of a platinum catalyst; or by dehydrogenation crosslinking condensation reaction between a diorganopolysiloxane containing hydroxyl end groups and a diorganopolysiloxane containing at least one hydrogen bonded to silicon, especially in the presence of an organotin; or by crosslinking condensation reaction of a diorganopolysiloxane containing hydroxyl end groups and of a hydrolysable organopolysiloxane; or by thermal crosslinking of organopolysiloxane, especially in the presence of an organoperoxide catalyst; or by crosslinking of organopolysiloxane via high-energy radiation such as gamma rays, ultraviolet rays or an electron beam.

Preferably, the organopolysiloxane elastomer is obtained by crosslinking addition reaction (A) of diorganopolysiloxane containing at least two hydrogens each bonded to a silicon, and (B) of diorganopolysiloxane containing at least two ethylenically unsaturated groups bonded to silicon, especially in the presence (C) of a platinum catalyst.

In particular, the organopolysiloxane elastomer may be obtained by reaction of a dimethylpolysiloxane with dimethylvinylsiloxy end groups and of methylhydrogenopolysiloxane with trimethylsiloxy end groups, in the presence of a platinum catalyst.

Compound (A) is the base reagent for the formation of organopolysiloxane elastomer, and the crosslinking is performed by addition reaction of compound (A) with compound (B) in the presence of the catalyst (C).

Compound (A) is in particular an organopolysiloxane containing at least two hydrogen atoms bonded to different silicon atoms in each molecule.

Compound (A) may have any molecular structure, in particular a linear-chain or branched-chain structure or a cyclic structure.

Compound (A) may have a viscosity at 25° C. ranging from 1 to 50 000 centistokes, in particular in order to be satisfactorily miscible with compound (B).

The organic groups bonded to the silicon atoms of compound (A) may be alkyl groups such as methyl, ethyl, propyl, butyl or octyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group.

Compound (A) may thus be chosen from methylhydrogenopolysiloxanes comprising trimethylsiloxy end groups, dimethylsiloxane-methylhydrogenosiloxane copolymers comprising trimethylsiloxy end groups, and dimethylsiloxane-methylhydrogenosiloxane cyclic copolymers.

Compound (B) is advantageously a diorganopolysiloxane containing at least two lower alkenyl groups (for example C₂-C₄); the lower alkenyl group may be chosen from vinyl, allyl and propenyl groups. These lower alkenyl groups may be located at any position on the organopolysiloxane molecule but are preferably located at the ends of the organopolysiloxane molecule. The organopolysiloxane (B) may have a branched-chain, linear-chain, cyclic or network structure but the linear-chain structure is preferred. Compound (B) may have a viscosity ranging from the liquid state to the gum state. Preferably, compound (B) has a viscosity of at least 100 centistokes at 25° C.

Besides the abovementioned alkenyl groups, the other organic groups bonded to the silicon atoms in compound (B) may be alkyl groups such as methyl, ethyl, propyl, butyl or octyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group.

The organopolysiloxanes (B) may be chosen from methylvinylpolysiloxanes, methylvinylsiloxane-dimethylsiloxane copolymers, dimethylpolysiloxanes comprising dimethylvinylsiloxy end groups, dimethylsiloxane-methylphenylsiloxane copolymers comprising dimethylvinylsiloxy end groups, dimethylsiloxane-diphenylsiloxane-methylvinylsiloxane copolymers comprising dimethylvinylsiloxy end groups, dimethylsiloxane-methylvinylsiloxane copolymers comprising trimethylsiloxy end groups, dimethylsiloxane-methylphenylsiloxane-methylvinylsiloxane copolymers comprising trimethylsiloxy end groups, methyl(3,3,3-trifluoropropyl)polysiloxanes comprising dimethylvinylsiloxy end groups, and dimethylsiloxane-methyl(3,3,3-trifluoropropyl)siloxane copolymers comprising dimethylvinylsiloxy end groups.

In particular, the organopolysiloxane elastomer may be obtained by reaction of a dimethylpolysiloxane with dimethylvinylsiloxy end groups and of methylhydrogenopolysiloxane with trimethylsiloxy end groups, in the presence of a platinum catalyst.

According to another alternative form, compound (B) may be an unsaturated hydrocarbon-based compound containing at least two lower alkenyl groups (for example C₂-C₄); the lower alkenyl group may be chosen from vinyl, allyl and propenyl groups. These lower alkenyl groups may be located in any position of the molecule, but are preferably located at the ends. By way of example, mention may be made of hexadiene, in particular of 1,5-hexadiene.

Advantageously, the sum of the number of ethylenic groups per molecule of compound (B) and of the number of hydrogen atoms bonded to silicon atoms per molecule of compound (A) is at least 5.

It is advantageous for compound (A) to be added in an amount such that the molecular ratio of the total amount of hydrogen atoms bonded to silicon atoms in compound (A) to the total amount of all the ethylenically unsaturated groups in compound (B) is within the range from 1.5/1 to 20/1.

Compound (C) is the catalyst for the crosslinking reaction, and is especially chloroplatinic acid, chloroplatinic acid-olefin complexes, chloroplatinic acid-alkenylsiloxane complexes, chloroplatinic acid-diketone complexes, platinum black and platinum on a support.

The catalyst (C) is preferably added in an amount of from 0.1 to 1000 parts by weight and better still from 1 to 100 parts by weight, as clean platinum metal, per 1000 parts by weight of the total amount of compounds (A) and (B).

The elastomer is advantageously a non-emulsifying elastomer.

The term “non-emulsifying” defines organopolysiloxane elastomers not containing any hydrophilic chains, and in particular not containing any polyoxyalkylene units (especially polyoxyethylene or polyoxypropylene) or any polyglyceryl units.

The organopolysiloxane elastomer particles are preferably conveyed in the form of a gel formed from an elastomeric organopolysiloxane included in at least one hydrocarbon-based oil and/or one silicone oil, as defined below. In these gels, the organopolysiloxane particles may be spherical or non-spherical particles.

Spherical non-emulsifying elastomers that may be used include, for example, those sold under the names DC 9040, DC 9041, DC 9509 and DC 9505 by the company Dow Corning.

Mention may also be made of those sold under the names KSG-6, KSG-15, KSG-16, KSG-18, KSG-41, KSG-42, KSG-43 and KSG-44 by the company Shin-Etsu; Gransil SR 5CYC Gel, Gransil SR DMF 10 Gel and Gransil SR DC556 Gel from the company Gransil RPS from Grant Industries; 1229-02-167, 1229-02-168 and SFE 839 from the company General Electric.

According to a preferred embodiment, the composition according to the invention comprises, as organopolysiloxane elastomer conveyed in an oil, a non-emulsifying elastomer, preferably spherical, preferably chosen from the compounds sold under the names DC 9040, DC 9041, DC 9509 and DC 9505 by the company Dow Corning.

According to one particular embodiment, elastomers may be used in a mixture with a cyclic silicone oil. An example that may be mentioned is the mixture of crosslinked organopolysiloxane/cyclopentasiloxane or a mixture of crosslinked organopolysiloxane/cyclohexasiloxane, for instance Gransil RPS D5 or Gransil RPS D6 from the company Grant Industries.

Emulsifying Organopolysiloxane Elastomer

According to another embodiment, the composition according to the invention comprises, as organopolysiloxane elastomer conveyed in an oil, an emulsifying elastomer.

The term “emulsifying organopolysiloxane elastomer” means an organopolysiloxane elastomer comprising at least one hydrophilic chain, such as polyoxyalkylenated organopolysiloxane elastomers and polyglycerolated silicone elastomers.

The emulsifying organopolysiloxane elastomer may be chosen from polyoxyalkylenated organopolysiloxane elastomers.

The polyoxyalkylenated organopolysiloxane elastomer is a crosslinked organopolysiloxane elastomer that may be obtained by crosslinking addition reaction of diorganopolysiloxane containing at least one hydrogen bonded to silicon and of a polyoxyalkylene containing at least two ethylenically unsaturated groups.

Preferably, the polyoxyalkylenated organopolysiloxane elastomer is obtained by crosslinking addition reaction (A1) of diorganopolysiloxane containing at least two hydrogens each bonded to a silicon, and (B1) of polyoxyalkylene containing at least two ethylenically unsaturated groups, especially in the presence (C1) of a platinum catalyst, as described, for instance, in patents U.S. Pat. No. 5,236,986 and U.S. Pat. No. 5,412,004.

In particular, the organopolysiloxane may be obtained by reaction of polyoxyalkylene (especially polyoxyethylene and/or polyoxypropylene) with dimethylvinylsiloxy end groups and of methylhydrogenopolysiloxane with trimethylsiloxy end groups, in the presence of a platinum catalyst.

The organic groups bonded to the silicon atoms of compound (A1) may be alkyl groups containing from 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl or 3,3,3-trifluoropropyl; aryl groups such as phenylethyl; and substituted monovalent hydrocarbon-based groups such as an epoxy group, a carboxylate ester group or a mercapto group.

Compound (A1) may thus be chosen from methylhydrogenopolysiloxanes containing trimethylsiloxy end groups, dimethylsiloxane-methylhydrogenosiloxane copolymers containing trimethylsiloxy end groups, dimethylsiloxane-methylhydrogenosiloxane cyclic copolymers, dimethylsiloxane-methylhydrogenosiloxane-laurylmethylsiloxane copolymers containing trimethylsiloxy end groups.

Compound (C1) is the catalyst for the crosslinking reaction, and is especially chloroplatinic acid, chloroplatinic acid-olefin complexes, chloroplatinic acid-alkenylsiloxane complexes, chloroplatinic acid-diketone complexes, platinum black and platinum on a support.

Advantageously, the polyoxyalkylenated organopolysiloxane elastomers may be formed from divinyl compounds, in particular polyoxyalkylenes containing at least two vinyl groups, which react with Si-H bonds of a polysiloxane.

Polyoxyalkylenated elastomers are described especially in patents

U.S. Pat. No. 5,236,986, U.S. Pat. No. 5,412,004, U.S. Pat. No. 5,837,793 and U.S. Pat. No. 5,811,487, the contents of which are incorporated by reference.

Polyoxyalkylenated organopolysiloxane elastomers that may be used include those sold under the names KSG-21, KSG-20, KSG-30, KSG-31, KSG-32, KSG-33, KSG-210, KSG-310, KSG-320, KSG-330 and KSG-340 by the company Shin-Etsu, and DC9010 and DC9011 by the company Dow Corning.

The emulsifying organopolysiloxane elastomer may also be chosen from polyglycerolated organopolysiloxane elastomers.

The polyglycerolated organopolysiloxane elastomer according to the invention is an organopolysiloxane elastomer that may be obtained by crosslinking addition reaction of diorganopolysiloxane containing at least one hydrogen bonded to silicon and of polyglycerolated compounds containing ethylenically unsaturated groups, especially in the presence of a platinum catalyst.

Preferably, the organopolysiloxane elastomer is obtained by crosslinking addition reaction (A2) of diorganopolysiloxane containing at least two hydrogens each bonded to a silicon, and (B2) of glycerolated compounds containing at least two ethylenically unsaturated groups, especially in the presence (C2) of a platinum catalyst.

In particular, the organopolysiloxane may be obtained by reaction of a polyglycerolated compound with dimethylvinylsiloxy end groups and of methylhydrogenopolysiloxane with trimethylsiloxy end groups, in the presence of a platinum catalyst.

Compound (A2) is the base reagent for the formation of an organopolysiloxane elastomer, and the crosslinking is performed by addition reaction of compound (A2) with compound (B2) in the presence of the catalyst (C2).

Compound (A2) is in particular an organopolysiloxane containing at least two hydrogen atoms bonded to different silicon atoms in each molecule.

Compound (A2) may have any molecular structure, especially a linear-chain or branched-chain structure or a cyclic structure.

Compound (A2) may have a viscosity at 25° C. ranging from 1 to 50 000 centistokes, especially so as to be satisfactorily miscible with compound (B2).

The organic groups bonded to the silicon atoms in compound (A2) may be alkyl groups containing from 1 to 18 carbon atoms, such as methyl, ethyl, propyl, butyl, octyl, decyl, dodecyl (or lauryl), myristyl, cetyl or stearyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon-based groups such as an epoxy group, a carboxylate ester group or a mercapto group.

Preferably, the said organic group is chosen from methyl, phenyl and lauryl groups.

Compound (A2) may thus be chosen from methylhydrogenopolysiloxanes containing trimethylsiloxy end groups, dimethylsiloxane-methylhydrogenosiloxane copolymers containing trimethylsiloxy end groups, dimethylsiloxane-methylhydrogenosiloxane cyclic copolymers and dimethylsiloxane-methylhydrogenosiloxane-laurylmethylsiloxane copolymers containing trimethylsiloxy end groups.

Compound (B2) may be a polyglycerolated compound corresponding to formula (B′) below:

CₘH₂ₘ₋₁—O-[Gly]ₙ-CₘH₂ₘ₋₁ (B′)

in which m is an integer ranging from 2 to 6, n is an integer ranging from 2 to 200, preferably ranging from 2 to 100, preferably ranging from 2 to 50, preferably ranging from 2 to 20, preferably ranging from 2 to 10 and preferentially ranging from 2 to 5, and in particular n is equal to 3; Gly denotes:

—CH₂—CH(OH)—CH₂—O— or —CH₂—CH(CH₂OH)—O—

Advantageously, the sum of the number of ethylenic groups per molecule in compound (B2) and of the number of hydrogen atoms bonded to silicon atoms per molecule in compound (A2) is at least 4.

It is advantageous for compound (A2) to be added in an amount such that the molecular ratio between the total amount of hydrogen atoms bonded to silicon atoms in compound (A2) and the total amount of all the ethylenically unsaturated groups in compound (B2) is within the range from 1/1 to 20/1.

Compound (C2) is the catalyst for the crosslinking reaction, and is especially chloroplatinic acid, chloroplatinic acid-olefin complexes, chloroplatinic acid-alkenylsiloxane complexes, chloroplatinic acid-diketone complexes, platinum black and platinum on a support.

The catalyst (C2) is preferably added in an amount of from 0.1 to 1000 parts by weight and better still from 1 to 100 parts by weight, as clean platinum metal, per 1000 parts by weight of the total amount of compounds (A2) and (B2).

The polyglycerolated organopolysiloxane elastomer is conveyed in gel form in at least one hydrocarbon-based oil and/or one silicone oil. In these gels, the polyglycerolated elastomer is often in the form of non-spherical particles.

Polyglycerolated organopolysiloxane elastomers that may be used include those sold under the names KSG-710, KSG-810, KSG-820, KSG-830 and KSG-840 by the company Shin-Etsu.

Preferably, the silicone elastomer conveyed in a first oil is non-emulsifying and is preferably devoid of a hydrophilic chain and in particular devoid of polyoxyalkylene units and polyglyceryl units.

Advantageously, the organopolysiloxane elastomer under consideration according to the invention is chosen from spherical non-emulsifying organopolysiloxane elastomers, polyglycerolated organopolysiloxane elastomers and polyoxyalkylenated organopolysiloxane elastomers.

Advantageously, the organopolysiloxane elastomer under consideration according to the invention is chosen from spherical non-emulsifying organopolysiloxane elastomers.

More particularly, the organopolysiloxane elastomer is obtained by crosslinking addition reaction of diorganopolysiloxane (A) containing at least two hydrogens each bonded to a silicon, and of diorganopolysiloxane (B) containing at least two ethylenically unsaturated groups bonded to silicon, especially in the presence of a platinum catalyst (C).

The proportion of oil(s) conveying the elastomer may advantageously represent from 70% to 95% by weight of the mixture of oil(s) and elastomer.

Advantageously, the composition according to the invention comprises a content of organopolysiloxane elastomer(s), expressed as solids (i.e. without taking into account the non-volatile oil in which the elastomer is conveyed) in an oil, in a content of at least 6% by weight and preferably from 6% to 10% by weight, relative to the total weight of the composition.

Non-Phenyl Silicone Oils

The composition according to the invention also comprises at least one volatile or non-volatile, non-phenyl silicone oil.

The term “silicone oil” means an oil containing at least one silicon atom, and in particular containing Si—O groups.

The term “non-phenyl” specifies that the said oil does not comprise in its structure any phenyl radicals.

The term “non-volatile” is intended to mean an oil of which the vapour pressure at 25° C. and atmospheric pressure is non-zero and is less than 0.02 mmHg (2.66 Pa) and better still less than 10⁻³ mmHg (0.13 Pa).

Non-Volatile Non-Phenyl Silicone Oils

Representative examples of these non-volatile non-phenyl silicone oils which may be mentioned include polydimethylsiloxanes; alkyl dimethicones; vinylmethyl methicones; and also silicones modified with aliphatic groups and/or with functional groups such as hydroxyl, thiol and/or amine groups.

It should be noted that “dimethicone” (INCI name) corresponds to a poly(dimethylsiloxane) (chemical name).

The non-volatile non-phenyl silicone oil is preferably chosen from non-volatile dimethicone oils.

In particular, these oils can be chosen from the following non-volatile oils:
polydimethylsiloxanes (PDMSs),
PDMSs comprising aliphatic groups, in particular alkyl or alkoxy groups, which are pendent and/or at the end of the silicone chain, these groups each comprising from 2 to 24 carbon atoms. By way of example, mention may be made of the cetyl dimethicone sold under the commercial reference Abil Wax 9801 from Evonik Goldschmidt,
PDMSs comprising aliphatic groups, or functional groups such as hydroxyl, thiol and/or amine groups,
polyalkylmethylsiloxanes substituted with functional groups such as hydroxyl, thiol and/or amine groups,
polysiloxanes modified with fatty acids, fatty alcohols or polyoxyalkylenes, and mixtures thereof.

Preferably, these non-volatile non-phenyl silicone oils are chosen from polydimethylsiloxanes; alkyl dimethicones and also PDMSs comprising aliphatic groups, in particular C₂-C₂₄ alkyl groups, and/or functional groups such as hydroxyl, thiol and/or amine groups.

The non-phenyl silicone oil may be chosen in particular from silicones of formula (I):

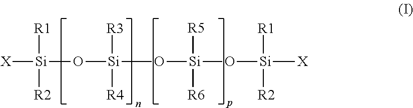

in which:

R₁, R₂, R₅ and R₆ are, together or separately, an alkyl radical containing from 1 to 6 carbon atoms,

R₃ and R₄ are, together or separately, an alkyl radical containing from 1 to 6 carbon atoms, a vinyl radical, an amine radical or a hydroxyl radical,

X is an alkyl radical containing from 1 to 6 carbon atoms, a hydroxyl radical or an amine radical,

n and p are integers chosen so as to have a fluid compound, in particular of which the viscosity at 25° C. is between 9 centistokes (cSt) (9×10⁻⁶ m²/s) and 800 000 cSt.

As non-volatile non-phenyl silicone oils which can be used according to the invention, mention may be made of those for which:
the substituents R₁ to R₆ and X represent a methyl group, and p and n are such that the viscosity is 500 000 cSt, for example the product sold under the name SE30 by the company General Electric, the product sold under the name AK 500000 by the company Wacker, the product sold under the name Mirasil DM 500 000 by the company Bluestar, and the product sold under the name Dow Corning 200 Fluid 500 000 cSt by the company Dow Corning,
the substituents R₁ to R₆ and X represent a methyl group, and p and n are such that the viscosity is 60 000 cSt, for example the product sold under the name Dow Corning 200 Fluid 60 000 CS by the company Dow Corning, and the product sold under the name Wacker Belsil DM 60 000 by the company Wacker,
the substituents R₁ to R₆ and X represent a methyl group, and p and n are such that the viscosity is 100 cSt or 350 cSt, for example the products sold respectively under the names Belsil DM100 and Dow Corning 200 Fluid 350 CS by the company Dow Corning,
the substituents R₁ to R₆ represent a methyl group, the group X represents a hydroxyl group, and n and p are such that the viscosity is 700 cSt, for example the product sold under the name Baysilone Fluid T0.7 by the company Momentive.

### Non-Phenyl Volatile Silicone Oils

According to one embodiment, the volatile oil is a silicone oil and may be chosen in particular from silicone oils with a flash point ranging from 40° C. to 102° C., preferably with a flash point of greater than 55° C. and less than or equal to 95° C., and preferentially ranging from 65° C. to 95° C.

As volatile silicone oils that may be used in the invention, mention may be made of linear or cyclic silicone oils, containing from 2 to 10 silicon atoms, optionally alkyl or alkoxy groups containing from 1 to 10 carbon atoms, and advantageously having a viscosity at 25° C. of less than 8 centistokes (cSt) (8×10⁻⁶ m²/s).

As volatile silicone oils that may be used in the invention, mention may be made especially of dimethicones with viscosities of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

In accordance with a particular embodiment of the invention, the content of volatile or non-volatile, non-phenyl silicone oil(s) ranges from 20% to 95% by weight, preferably between 35% and 85% by weight and even more preferably from 40% to 80% by weight, relative to the weight of the composition.

Apolar Hydrocarbon-Based Compounds

The composition according to the invention also comprises at least one apolar hydrocarbon-based compound, which is liquid or solid at room temperature,

Preferably, this compound is non-volatile.

Thus, the composition may comprise at least one apolar hydrocarbon-based non-volatile oil, at least one apolar hydrocarbon-based wax, or mixtures thereof.

Apolar Non-Volatile Hydrocarbon-Based Oils

The term “hydrocarbon-based oil” means an oil formed essentially from, or even constituted by, carbon and hydrogen atoms, and optionally oxygen and nitrogen atoms, and not containing any silicon or fluorine atoms. It may contain alcohol, ester, ether, carboxylic acid, amine and/or amide groups.

These hydrocarbon-based oils may be of plant, mineral or synthetic origin.

For the purposes of the present invention, the term “apolar oil” is intended to mean an oil of which the solubility parameter at 25° C., δₐ, is equal to 0 (J/cm³)^{1/2}.

The definition and calculation of the solubility parameters in the Hansen three-dimensional solubility space are described in the article by C. M. Hansen: The three-dimensional solubility parameters, J. Paint Technol. 39, 105 (1967).

According to this Hansen space:
δ_{D} characterizes the London dispersion forces derived from the formation of dipoles induced during molecular impacts;
δₚ characterizes the Debye interaction forces between permanent dipoles and also the Keesom interaction forces between induced dipoles and permanent dipoles;
δₕ characterizes the specific interaction forces (such as hydrogen bonding, acid/base, donor/acceptor, etc.); and
δ_{q} is determined by the equation: δₐ=(δₚ²+δₕ²)^{1/2}.

The parameters δₚ, δₕ, δ_{D} and δₐ are expressed in (J/cm³)^{1/2}.

Preferably, the non-volatile apolar hydrocarbon-based oil may be chosen from linear or branched hydrocarbons of mineral or synthetic origin, such as:
liquid paraffin or derivatives thereof,
squalane,
isoeicosane,
naphthalene oil,
polybutenes, hydrogenated polybutenes, such as Indopol H-100 (molar mass or MW=965 g/mol), Indopol H-300 (MW=1340 g/mol) and Indopol H-1500 (MW=2160 g/mol) sold or manufactured by the company Amoco,
polyisobutenes, hydrogenated polyisobutenes, such as Parleam® sold by the company Nippon Oil Fats, Panalane H-300 E sold or manufactured by the company Amoco (MW=1340 g/mol), Viseal 20000 sold or manufactured by the company Synteal (MW=6000 g/mol) and Rewopal PIB 1000 sold or manufactured by the company Witco (MW=1000 g/mol), or alternatively Parleam Lite sold by NOF Corporation,
polydecenes and hydrogenated polydecenes such as: Puresyn 10 (MW=723 g/mol) and Puresyn 150 (MW=9200 g/mol) sold or manufactured by the company Mobil Chemicals, or alternatively Puresyn 6 sold by ExxonMobil Chemical),
decene/butene copolymers and polybutene/polyisobutene copolymers, in particular Indopol L-14,
and mixtures thereof.

Preferably, the composition according to the invention comprises at least one apolar oil preferably chosen from; hydrogenated or non-hydrogenated polybutenes or polyisobutenes; hydrogenated or non-hydrogenated polydecenes; and mixtures thereof.

Apolar Hydrocarbon-Based Waxes

The wax(es) under consideration in the context of the present invention are generally lipophilic compounds that are solid at 25° C., with a solid/liquid reversible change of state, having a melting point of greater than or equal to 30° C., which may be up to 200° C. and especially up to 120° C.

In particular, the waxes that are suitable for the invention may have a melting point of greater than or equal to 45° C. and in particular greater than or equal to 55° C.

Preferably, the waxes have an heat of fusion Δ For the purposes of the invention, the melting point corresponds to the temperature of the most endothermic peak observed on thermal analysis (DSC) as described in standard ISO 11357-3; 1999. The melting point of the wax may be measured using a differential scanning calorimeter (DSC), for example the calorimeter sold under the name DSC Q2000 by the company TA Instruments.

Hf of greater than or equal to 70 J/g.

Preferably, the waxes comprise at least one crystallizable part, which is visible by X-ray observation.

The Measuring Protocol is as Follows:

A sample of 5 mg of wax placed in a crucible is subjected to a first temperature rise ranging from −20° C. to 120° C., at a heating rate of 10° C./minute, it is then cooled from 120° C. to −20° C. at a cooling rate of 10° C./minute and is finally subjected to a second temperature increase ranging from −20° C. to 120° C. at a heating rate of 5° C./minute. During the second temperature increase, the following parameters are measured:
the melting point (T_{f}) of the wax, as mentioned previously corresponding to the temperature of the most endothermic peak of the melting curve observed, representing the variation of the difference in power absorbed as a function of the temperature, and
ΔHf: the heat of fusion of the wax, corresponding to the integral entire melting curve obtained. This heat of fusion of the wax is the amount of energy required to make the compound change from the solid state to the liquid state. It is expressed in J/g.

The wax may especially have a hardness ranging from 0.05 MPa to 15 MPa and preferably ranging from 6 MPa to 15 MPa. The hardness is determined by measuring the compression force, measured at 20° C. using the texturometer sold under the name TA-TX2i by the company Rheo, equipped with a stainless-steel cylinder 2 mm in diameter travelling at a measuring speed of 0.1 mm/s, and penetrating the wax to a penetration depth of 0.3 mm.

For the purposes of the present invention, the term “apolar wax” means a wax whose solubility parameter at 25° C. as defined below, δₐ, is equal to 0 (J/cm³)^{1/2}.

Apolar waxes are in particular hydrocarbon-based waxes constituted solely of carbon and hydrogen atoms, and free of heteroatoms such as N, O, Si and P.

The term “hydrocarbon-based wax” means a wax formed essentially from, or even constituted of, carbon and hydrogen atoms, and optionally oxygen and nitrogen atoms, and that does not contain any silicon or fluorine atoms. It may contain alcohol, ester, ether, carboxylic acid, amine and/or amide groups.

More particularly, the apolar wax may be chosen from microcrystalline waxes, paraffin waxes, ozokerite, polyethylene waxes, polymethylene waxes and microwaxes, and mixtures thereof.

As microcrystalline waxes that may be used, mention may be made of Multiwax W 445® sold by the company Sonneborn, and Microwax HW® and Base Wax 30540® sold by the company Paramelt.

An ozokerite that may be mentioned is Ozokerite Wax SP 1020 P.

Polyethylene waxes that may be mentioned include Performalene 500-L Polyethylene and Performalene 400 Polyethylene sold by New Phase Technologies.

Polymethylene waxes that may be mentioned include the Polymethylene Wax (54° C.) sold under the reference Cirebelle 303 and the Polymethylene Wax (80° C.) sold under the reference Cirebelle 108, sold by Cirebelle.

As microwaxes that may be used in the compositions according to the invention as apolar wax, mention may be made especially of polyethylene microwaxes such as those sold under the names Micropoly 200®, 220®, 220L® and 250S® by the company Micro Powders.

More particularly, the content of apolar hydrocarbon-based compound, which is liquid or solid at room temperature, ranges from 2% to 50% by weight, more particularly from 2% to 30% by weight and more preferably between 2% and 20% by weight relative to the weight of the composition.

It should be noted that if the composition comprises, as apolar hydrocarbon-based compound, at least one wax, the content of this type of solid compound remains between 5% and 15% by weight, relative to the weight of the composition.

Pasty Fatty Substances

The composition according to the invention may also comprise at least one pasty fatty substance.

For the purposes of the present invention, the term “pasty fatty substance” means a lipophilic fatty compound that undergoes a reversible solid/liquid change in state, which exhibits, in the solid state, an anisotropic crystalline arrangement and which comprises, at a temperature of 23° C., a liquid fraction and a solid fraction.

In other words, the starting melting point of the pasty fatty substance can be less than 23° C. The liquid fraction of the pasty fatty substance measured at 23° C. can represent from 9% to 97% by weight of the pasty fatty substance. This liquid fraction at 23° C. preferably represents between 15% and 85% and more preferably between 40% and 85% by weight.

For the purposes of the invention, the melting point corresponds to the temperature of the most endothermic peak observed in thermal analysis (DSC) as described in the standard ISO 11357-3; 1999. The melting point of a pasty fatty substance may be measured using a differential scanning calorimeter (DSC), for example the calorimeter sold under the name MDSC 2920 by the company TA Instruments.

The Measurement Protocol is as Follows:

A sample of 5 mg of pasty fatty substance placed in a crucible is subjected to a first temperature rise ranging from −20° C. to 100° C., at a heating rate of 10° C./minute, is then cooled from 100° C. to −20° C. at a cooling rate of 10° C./minute and is finally subjected to a second temperature rise ranging from −20° C. to 100° C. at a heating rate of 5° C./minute. During the second temperature rise, the variation in the difference in power absorbed by the empty crucible and by the crucible containing the sample of pasty fatty substance is measured as a function of the temperature. The melting point of the pasty fatty substance is the value of the temperature corresponding to the tip of the peak of the curve representing the variation in the difference in power absorbed as a function of the temperature.

The liquid fraction by weight of the pasty fatty substance at 23° C. is equal to the ratio of the heat of fusion consumed at 23° C. to the heat of fusion of the pasty fatty substance.

The heat of fusion of the pasty fatty substance is the heat consumed by the pasty fatty substance in order to pass from the solid state to the liquid state. The pasty fatty substance is said to be in the solid state when all of its mass is in crystalline solid form. The pasty fatty substance is said to be in the liquid state when all of its mass is in liquid form.

The heat of fusion of the pasty fatty substance is equal to the area under the curve of the thermogram obtained using a differential scanning calorimeter (DSC), such as the calorimeter sold under the name MDSC 2920 by the company TA Instrument, with a temperature rise of 5° C. or 10° C. per minute, according to the standard ISO 11357-3; 1999.

The heat of fusion of the pasty fatty substance is the amount of energy required to make the pasty fatty substance change from the solid state to the liquid state. It is expressed in J/g.

The heat of fusion consumed at 23° C. is the amount of energy absorbed by the sample to change from the solid state to the state that it has at 23° C., composed of a liquid fraction and a solid fraction.

The liquid fraction of the pasty fatty substance measured at 32° C. preferably represents from 30% to 100% by weight of the pasty fatty substance, preferably from 50% to 100%, more preferably from 60% to 100% by weight of the pasty fatty substance. When the liquid fraction of the pasty fatty substance measured at 32° C. is equal to 100%, the temperature of the end of the melting range of the pasty fatty substance is less than or equal to 32° C.

The liquid fraction of the pasty fatty substance measured at 32° C. is equal to the ratio of the heat of fusion consumed at 32° C. to the heat of fusion of the pasty fatty substance. The heat of fusion consumed at 32° C. is calculated in the same way as the heat of fusion consumed at 23° C.

The pasty fatty substance may in particular be chosen from synthetic fatty substances and fatty substances of vegetable origin.

Preferably, the pasty fatty substances that are suitable for use in the invention are chosen from hydrocarbon-based compounds and comprise, besides carbon and hydrogen atoms, at least oxygen atoms.

The pasty fatty substance(s) may be chosen in particular from:
lanolin and derivatives thereof, such as lanolin alcohol, oxyethylenated lanolins, acetylated lanolin, lanolin esters such as isopropyl lanolate, and oxypropylenated lanolins,
petroleum jelly (also known as petrolatum),
polyol ethers chosen from pentaerythritol and polyalkylene glycol ethers, fatty alcohol ethers and sugar ethers, and mixtures thereof, the pentaerythrityl and polyethylene glycol ether comprising 5 oxyethylene units (5 OE) (CTFA name: PEG-5 Pentaerythrityl Ether), polypropylene glycol pentaerythrityl ether comprising five oxypropylene (5 OP) units (CTFA name: PEG-5 Pentaerythrityl Ether) and mixtures thereof, and more especially the mixture PEG-5 Pentaerythrityl Ether, PPG-5 Pentaerythrityl Ether and soybean oil, sold under the name Lanolide by the company Vevy, which is a mixture in which the constituents are in a 46/46/8 weight ratio: 46% PEG-5 Pentaerythrityl Ether, 46% PPG-5 Pentaerythrityl Ether and 8% soybean oil,
liposoluble polyethers resulting from the polyetherification between one or more C₂-C₁₀₀ and preferably C₂-C₅₀ diols,

Among the liposoluble polyethers that are particularly considered are copolymers of ethylene oxide and/or of propylene oxide with long-chain C₆-C₃₀ alkylene oxides, more preferably such that the weight ratio of the ethylene oxide and/or propylene oxide to alkylene oxides in the copolymer is from 5:95 to 70:30. In this family, mention will be made especially of copolymers such as the long-chain alkylene oxides arranged in blocks having an average molecular weight from 1000 to 10 000, for example a polyoxyethylene/polydodecyl glycol block copolymer such as the ethers of dodecanediol (22 mol) and of polyethylene glycol (45 OE) sold under the brand name Elfacos ST9 by Akzo Nobel.
esters and polyesters.

Among the esters, the following are especially considered:
esters of a glycerol oligomer, especially diglycerol esters, in particular condensates of adipic acid and of glycerol, for which some of the hydroxyl groups of the glycerols have reacted with a mixture of fatty acids such as stearic acid, capric acid, isostearic acid and 12-hydroxystearic acid, such as, for example, bis-diglyceryl polyacyladipate-2 sold under the reference Softisan® 649 by the company Sasol,
vinyl ester homopolymers containing C₈-C₃₀ alkyl groups, such as polyvinyl laurate (sold especially under the reference Mexomer PP by the company Chimex),
the arachidyl propionate sold under the brand name Waxenol 801 by Alzo,
phytosterol esters,
fatty acid triglycerides and derivatives thereof,
pentaerythritol esters,
esters of a diol dimer and of a diacid dimer, where appropriate esterified on their free alcohol or acid function(s) with acid or alcohol radicals, especially dimer dilinoleate esters; such esters may be chosen especially from the esters having the following INCI nomenclature: bis-behenyl/isostearyl/phytosteryl dimer dilinoleyl dimer dilinoleate (Plandool G), phytosteryl/isostearyl/cetyl/stearyl/behenyl dimer dilinoleate (Plandool H or Plandool S), and mixtures thereof,
butters of plant origin, such as mango butter, such as the product sold under the name Lipex 203 by the company Aarhuskarlshamn, shea butter, in particular the product whose INCI name is Butyrospermum Parkii Butter, such as the product sold under the reference Sheasoft® by the company Aarhuskarlshamn, cupuacu butter (Rain Forest RF3410 from the company Beraca Sabara), murumuru butter (Rain Forest RF3710 from the company Beraca Sabara), cocoa butter; and also orange wax, for instance the product sold under the reference Orange Peel Wax by the company Koster Keunen,
totally or partially hydrogenated plant oils, for instance hydrogenated soybean oil, hydrogenated coconut oil, hydrogenated rapeseed oil, mixtures of hydrogenated plant oils such as the mixture of hydrogenated soybean, coconut, palm and rapeseed plant oil, for example the mixture sold under the reference Akogel® by the company Aarhuskarlshamn (INCI name Hydrogenated Vegetable Oil), the trans-isomerized partially hydrogenated jojoba oil manufactured or sold by the company Desert Whale under the commercial reference Iso-Jojoba-500, partially hydrogenated olive oil, for instance the compound sold under the reference Beurrolive by the company Soliance,
hydrogenated castor oil esters, such as hydrogenated castor oil dimer dilinoleate, for example Risocast DA-L sold by Kokyu Alcohol Kogyo, and hydrogenated castor oil isostearate, for example Salacos HCIS (V-L) sold by Nisshin Oil,
and mixtures thereof.

More particularly, the pasty fatty substance(s) are chosen from lanolin and derivatives thereof, polyol ethers, liposoluble polyethers, esters and polyesters, and mixtures thereof.

According to a preferred embodiment, the pasty fatty substance(s) are chosen from esters and in particular butters of plant origin, and totally or partially hydrogenated plant oils, and mixtures thereof.

Preferably, if the composition comprises at least one pasty fatty substance, then the content thereof represents from 0.1% to 25% by weight and preferably from 10% to 20% by weight relative to the total weight of the composition.

Additional Volatile Oils

According to a particular embodiment of the invention, the composition may also comprise at least one additional volatile oil other than the silicone volatile oil mentioned previously.

Preferably, the additional volatile oil is chosen from hydrocarbon-based oils, which are preferably apolar, or a fluoro oil.

Volatile fluoro oils that may be mentioned include nonafluoromethoxybutane and perfluoromethylcyclopentane, and mixtures thereof.

As regards the apolar volatile hydrocarbon-based oils, they may have a flash point ranging from 40° C. to 102° C., preferably ranging from 40° C. to 55° C. and preferentially ranging from 40° C. to 50° C.

The hydrocarbon-based volatile oil(s) may be chosen especially from hydrocarbon-based volatile oils containing from 8 to 16 carbon atoms, and mixtures thereof, and especially:
branched C₈-C₁₆ alkanes such as C₈-C₁₆ isoalkanes (also known as isoparaffins), isododecane, isodecane and isohexadecane, and, for example, the oils sold under the trade name Isopar or Permethyl,
linear alkanes, for instance n-dodecane (C12) and n-tetradecane (C14) sold by Sasol under the respective references Parafol 12-97 and Parafol 14-97, and also mixtures thereof, the undecane-tridecane mixture (Cetiol UT), the mixtures of n-undecane (C11) and of n-tridecane (C13) obtained in Examples 1 and 2 of patent application WO 2008/155 059 from the company Cognis, and mixtures thereof.

Preferably, the additional volatile oil, if it is present, is chosen from apolar hydrocarbon-based volatile oils.

When the composition comprises at least one volatile oil, the content thereof more particularly represents from 2% to 50% by weight, more particularly from 2% to 30% by weight and preferably from 2% to 20% by weight relative to the total weight of the said composition.

Fillers

A composition according to the invention may also contain at least one or more filler(s).

The term “fillers” should be understood as meaning colourless or white, mineral or synthetic particles of any shape, which are insoluble in the medium of the composition, irrespective of the temperature at which the composition is manufactured. These fillers serve in particular to modify the rheology or the texture of the composition.

The fillers may be mineral or organic and of any shape, platelet-shaped, spherical or oblong, irrespective of the crystallographic form (for example lamellar, cubic, hexagonal, orthorhombic, etc.). Mention may be made of talc, mica, silica, kaolin, bentone, fumed silica particles, optionally hydrophilic- or hydrophobic-treated, polyamide (Nylon®) powder (Orgasol® from Atochem), poly-β-alanine powder and polyethylene powder, tetrafluoroethylene polymer (Teflon®) powders, lauroyllysine, starch, boron nitride, hollow polymer microspheres such as polyvinylidene chloride/acrylonitrile microspheres, for instance Expancel® (Nobel Industrie), acrylic acid copolymer microspheres (Polytrap® from the company Dow Corning) and silicone resin microbeads (for example Tospearls® from Toshiba), precipitated calcium carbonate, magnesium carbonate, magnesium hydrogen carbonate, hydroxyapatite, hollow silica microspheres (Silica Beads® from Maprecos), elastomeric polyorganosiloxane particles, glass or ceramic microcapsules, and metal soaps derived from organic carboxylic acids containing from 8 to 22 carbon atoms and preferably from 12 to 18 carbon atoms, for example zinc stearate, magnesium stearate, lithium stearate, zinc laurate or magnesium myristate.

They may also be particles comprising a copolymer, the said copolymer comprising trimethylol hexyl lactone. In particular, it may be a copolymer of hexamethylene diisocyanate/trimethylol hexyl lactone. Such particles are especially commercially available, for example, under the name Plastic Powder D-400® or Plastic Powder D-800® from the company Toshiki.

Preferably, the composition contains between 0.01% and 25% by weight, in particular between 0.1% and 20% by weight and even more preferentially from 0.1% to 10% by weight, or even from 0.1% to 3% by weight of fillers relative to the total weight of the composition.

### Silica

According to a preferred embodiment, the composition comprises at least one filler, in particular chosen from fumed silica particles that have optionally been hydrophilic- or hydrophobic-treated, preferably hydrophobic-treated; hydrophobic silica aerogels. Preferably, the composition comprises at least one filler known as Silica Dimethyl Silylate (according to the CTFA).

The hydrophobic groups may especially be dimethylsilyloxyl or polydimethylsiloxane groups, which are especially obtained by treating fumed silica in the presence of polydimethylsiloxane or dimethyldichlorosilane. Silicas thus treated are known as Silica Dimethyl Silylate according to the CTFA (6th edition, 1995). They are sold, for example, under the references Aerosil R972® and Aerosil R974® by Degussa, and Cab-O-Sil TS-610® and Cab-O-Sil TS-720® by Cabot.

### Hydrophobic Silica Aerogels

Preferably, the composition according to the invention comprises at least hydrophobic silica aerogel particles.

Silica aerogels are porous materials obtained by replacing (by drying) the liquid component of a silica gel with air.

They are generally synthesized via a sol-gel process in a liquid medium and then dried, usually by extraction with a supercritical fluid, the one most commonly used being supercritical CO₂. This type of drying makes it possible to avoid shrinkage of the pores and of the material. The sol-gel process and the various drying operations are described in detail in Brinker C. J., and Scherer G. W., Sol-Gel Science, New York: Academic Press, 1990.

The hydrophobic silica aerogel particles used in the present invention have a specific surface area per unit of mass (S_{M}) ranging from 500 to 1500 m²/g, preferably from 600 to 1200 m²/g and better still from 600 to 800 m²/g, and a size expressed as the volume-average diameter (D[0.5]) ranging from 1 to 1500 μm, better still from 1 to 1000 μm, preferably from 1 to 100 μm, in particular from 1 to 30 μm, more preferably from 5 to 25 μm, better still from 5 to 20 μm and even better still from 5 to 15 μm.

According to one embodiment, the hydrophobic silica aerogel particles used in the present invention have a size, expressed as the volume-average diameter (D[0.5]), ranging from 1 to 30 μm, preferably from 5 to 25 μm, better still from 5 to 20 μm and even better still from 5 to 15 μm.

The specific surface area per unit of mass can be determined by the nitrogen absorption method, known as the BET (Brunauer-Emmett-Teller) method, described in The Journal of the American Chemical Society, vol. 60, page 309, February 1938, which corresponds to international standard ISO 5794/1 (appendix D). The BET specific surface area corresponds to the total specific surface area of the particles under consideration.

The sizes of the silica aerogel particles may be measured by static light scattering using a commercial particle size analyser such as the MasterSizer 2000 machine from Malvern. The data are processed on the basis of the Mie scattering theory. This theory, which is exact for isotropic particles, makes it possible to determine, in the case of non-spherical particles, an “effective” particle diameter. This theory is especially described in the publication by Van de Hulst, H. C., “Light Scattering by Small Particles”, Chapters 9 and 10, Wiley, New York, 1957.

According to an advantageous embodiment, the hydrophobic silica aerogel particles used in the present invention have a specific surface area per unit of mass (S_{M}) ranging from 600 to 800 m²/g and a size expressed as the volume-average diameter (D[0.5]) ranging from 5 to 20 μm and even better still from 5 to 15 μm.

The silica aerogel particles used in the present invention may advantageously have a tapped density p ranging from 0.02 g/cm³ to 0.10 g/cm³, preferably from 0.03 g/cm³ to 0.08 g/cm³ and preferably from 0.05 g/cm³ to 0.08 g/cm³.

In the context of the present invention, this density, known as the tapped density, may be assessed according to the following protocol:

40 g of powder are poured into a measuring cylinder; the measuring cylinder is then placed on the Stay 2003 machine from Stampf Volumeter; the measuring cylinder is subsequently subjected to a series of 2500 tapping actions (this operation is repeated until the difference in volume between 2 consecutive tests is less than 2%); and then the final volume Vf of tapped powder is measured directly on the measuring cylinder. The tapped density is determined by the ratio m/Vf, in this instance 40/Vf (Vf being expressed in cm³ and m in g).

According to a preferred embodiment, the hydrophobic silica aerogel particles used in the present invention have a specific surface area per unit of volume Sᵥ ranging from 5 to 60 m²/cm³, preferably from 10 to 50 m²/cm³ and better still from 15 to 40 m²/cm³.

The specific surface area per unit of volume is given by the relationship: S_{V}=S_{M}×ρ where ρ is the tapped density, expressed in g/cm³, and S_{M} is the specific surface per unit of mass, expressed in m²/g, as defined above.

Preferably, the hydrophobic silica aerogel particles according to the invention have an oil-absorbing capacity, measured at the wet point, ranging from 5 to 18 ml/g, preferably from 6 to 15 ml/g and better still from 8 to 12 ml/g.

The absorbing capacity measured at the wet point, noted Wp, corresponds to the amount of oil that needs to be added to 100 g of particles in order to obtain a homogeneous paste.

It is measured according to the “wet point” method or the method for determining the oil uptake of a powder described in standard NF T 30-022. It corresponds to the amount of oil adsorbed onto the available surface of the powder and/or absorbed by the powder by measurement of the wet point, described below:

An amount m=2 g of powder is placed on a glass plate, and the oil (isononyl isononanoate) is then added dropwise. After addition of 4 to 5 drops of oil to the powder, mixing is carried out using a spatula, and addition of oil is continued until conglomerates of oil and powder have formed. From this point, the oil is added at the rate of one drop at a time and the mixture is subsequently triturated with the spatula. The addition of oil is stopped when a firm, smooth paste is obtained. This paste must be able to be spread on the glass plate without cracking or forming lumps. The volume Vs (expressed in ml) of oil used is then noted.

The oil uptake corresponds to the ratio Vs/m.

The aerogels used according to the present invention are hydrophobic silica aerogels, preferably of silyl silica (INCI name: silica silylate).

The term “hydrophobic silica” is understood to mean any silica whose surface is treated with silylating agents, for example with halogenated silanes, such as alkylchlorosilanes, siloxanes, in particular dimethylsiloxanes, such as hexamethyldisiloxane, or silazanes, so as to functionalize the OH groups with silyl Si—Rn groups, for example trimethylsilyl groups.

As regards the preparation of hydrophobic silica aerogel particles that have been surface-modified by silylation, reference may be made to document U.S. Pat. No. 7,470,725.

Use will preferably be made of hydrophobic silica aerogel particles surface-modified with trimethylsilyl groups.

As hydrophobic silica aerogels that may be used in the invention, examples that may be mentioned include the aerogel sold under the name VM-2260 (INCI name: Silica silylate), by the company Dow Corning, the particles of which have an average size of about 1000 microns and a specific surface area per unit of mass ranging from 600 to 800 m²/g.

Mention may also be made of the aerogels sold by Cabot under the references Aerogel TLD 201, Aerogel OGD 201, Aerogel TLD 203, Enova® Aerogel MT 1100 and Enova Aerogel MT 1200.

Use will preferably be made of the aerogel sold under the name VM-2270 (INCI name: Silica silylate), by the company Dow Corning, the particles of which have an average size ranging from 5-15 microns and a specific surface area per unit of mass ranging from 600 to 800 m²/g.

Preferably, the silica or hydrophobic silica aerogel particles are present in the composition according to the invention in an active material content ranging from 0.1% to 10% by weight and preferably from 0.1% to 6% by weight relative to the total weight of the composition.

Preferably, the hydrophobic silica aerogel particles are present in the composition according to the invention in an active material content ranging from 0.2% to 4% by weight or even from 0.2% to 2% by weight relative to the total weight of the composition.

Dyestuffs

Although this does not correspond to a preferred embodiment of the invention, the composition may optionally comprise at least one dyestuff (also known as a colouring agent), which may be chosen from water-soluble or liposoluble dyes, pigments and nacres, and mixtures thereof.

The composition according to the invention may also comprise one or more dyestuffs chosen from water-soluble dyes and pulverulent dyestuffs, for instance pigments, nacres and glitter flakes that are well known to those skilled in the art.

The dyestuffs may be present in the composition in a content ranging from 0.01% to 30% by weight relative to the weight of the composition, preferably from 0.1% to 20% by weight relative to the weight of the composition and even more preferentially from 0.1% to 10% by weight relative to the weight of the composition.

The term “pigments” should be understood as meaning white or coloured, mineral or organic particles that are insoluble in an aqueous solution, which are intended to colour and/or opacify the resulting film.

The pigments may be present in a proportion of from 0.01% to 20% by weight, especially from 0.1% to 15% by weight and in particular from 0.2% to 10% by weight, relative to the total weight of the cosmetic composition.

As mineral pigments that may be used in the invention, mention may be made of titanium oxide, zirconium oxide or cerium oxide, and also zinc oxide, iron oxide or chromium oxide, ferric blue, manganese violet, ultramarine blue and chromium hydrate.

The pigment may also be a pigment having a structure that may be, for example, of sericite/brown iron oxide/titanium dioxide/silica type. Such a pigment is sold, for example, under the reference Coverleaf NS or JS by the company Chemicals and Catalysts, and has a contrast ratio in the region of 30.

The dyestuff may also comprise a pigment with a structure that may be, for example, of silica microsphere type containing iron oxide. An example of a pigment having this structure is the product sold by the company Miyoshi under the reference PC Ball PC-LL-100 P, this pigment consisting of silica microspheres containing yellow iron oxide.

Among the organic pigments that may be used in the invention, mention may be made of carbon black, pigments of D&C type, lakes based on cochineal carmine or on barium, strontium, calcium or aluminium, or alternatively the diketopyrrolopyrroles (DPPs) described in documents EP-A-542 669, EP-A-787 730, EP-A-787 731 and WO-A-96/08537.

The term “nacres” should be understood as meaning coloured particles of any form, which may or may not be iridescent, especially produced by certain molluscs in their shell, or alternatively synthesized, and which have a colour effect via optical interference.

The nacres may be chosen from nacreous pigments such as titanium mica coated with an iron oxide, titanium mica coated with bismuth oxychloride, titanium mica coated with chromium oxide, titanium mica coated with an organic dye and also nacreous pigments based on bismuth oxychloride. They may also be mica particles at the surface of which are superposed at least two successive layers of metal oxides and/or of organic dyestuffs.

Examples of nacres that may also be mentioned include natural mica coated with titanium oxide, with iron oxide, with natural pigment or with bismuth oxychloride.

Among the commercially available nacres that may be mentioned are the nacres Timica, Flamenco and Duochrome (on mica base) sold by the company Engelhard, the Timiron nacres sold by the company Merck, the Prestige nacres on mica base sold by the company Eckart and the Sunshine nacres on synthetic mica base sold by the company Sun Chemical.

The nacres may more particularly have a yellow, pink, red, bronze, orange, brown, gold and/or coppery colour or tint.

As illustrations of nacres that may be used in the context of the present invention, mention may be made especially of the gold-coloured nacres sold especially by the company Engelhard under the name Brilliant gold 212G (Timica), Gold 222C (Cloisonne), Sparkle gold (Timica), Gold 4504 (Chromalite) and Monarch gold 233X (Cloisonne); the bronze nacres sold especially by the company Merck under the name Bronze fine (17384) (Colorona) and Bronze (17353) (Colorona) and by the company Engelhard under the name Super bronze (Cloisonne); the orange nacres sold especially by the company Engelhard under the name Orange 363C (Cloisonne) and Orange MCR 101 (Cosmica) and by the company Merck under the name Passion orange (Colorona) and Matte orange (17449) (Microna); the brown nacres sold especially by the company Engelhard under the name Nu-antique copper 340XB (Cloisonne) and Brown CL4509 (Chromalite); the nacres with a copper tint sold especially by the company Engelhard under the name Copper 340A (Timica); the nacres with a red tint sold especially by the company Merck under the name Sienna fine (17386) (Colorona); the nacres with a yellow tint sold especially by the company Engelhard under the name Yellow (4502) (Chromalite); the red nacres with a gold tint sold especially by the company Engelhard under the name Sunstone G012 (Gemtone); the pink nacres sold especially by the company Engelhard under the name Tan opale G005 (Gemtone); the black nacres with a gold tint sold especially by the company Engelhard under the name Nu antique bronze 240 AB (Timica), the blue nacres sold especially by the company Merck under the name Matte blue (17433) (Microna), the white nacres with a silvery tint sold especially by the company Merck under the name Xirona Silver, and the golden-green pink-orange nacres sold especially by the company Merck under the name Indian summer (Xirona), and mixtures thereof.

The cosmetic composition according to the invention may also contain at least one material with a specific optical effect as dyestuff.

This effect is different from a simple conventional hue effect, i.e. a unified and stabilized effect as produced by standard dyestuffs, for instance monochromatic pigments. For the purposes of the invention, the term “stabilized” means lacking an effect of variability of the colour as a function of the angle of observation or alternatively in response to a temperature change.

For example, this material may be chosen from particles with a metallic glint, goniochromatic colouring agents, diffractive pigments, thermochromic agents, optical brighteners, and also fibres, especially interference fibres. Needless to say, these various materials may be combined so as to afford the simultaneous manifestation of two effects, or even of a novel effect in accordance with the invention.

The term “dyes” should be understood as meaning compounds that are generally organic, which are soluble in fatty substances such as oils or in an aqueous-alcoholic phase.

The cosmetic composition according to the invention may also comprise water-soluble or liposoluble dyes. The liposoluble dyes are, for example, Sudan red, DC Red 17, DC Green 6, β-carotene, Sudan brown, DC Yellow 11, DC Violet 2, DC Orange 5 and quinoline yellow. The water-soluble dyes are, for example, beetroot juice or methylene blue.

If the composition comprises any, the content of dyestuff(s) represents from 0.1% to 20% by weight and more specifically from 0.1% to 10% by weight relative to the total weight of the composition.

Additives

A composition according to the invention may furthermore comprise any ingredient conventionally used as additive in cosmetics and dermatology.

These additives are advantageously chosen from antioxidants, thickeners, sweeteners, basifying or acidifying preserving agents, and mixtures thereof, and may be chosen advantageously from those proposed in Table 1 of the Codex Alimentarius.

As antioxidant, a composition in accordance with the invention may advantageously comprise at least one pentaerythrityl di-t-butyl hydroxycinnamate.

A composition according to the invention may also contain flavourings and/or fragrances.

Needless to say, those skilled in the art will take care to select this or these optional additional compound(s), and/or the amount thereof, such that the advantageous properties of the composition according to the invention are not, or are not substantially, adversely affected by the envisaged addition.

The examples that follow illustrate the invention without limiting the scope thereof.

### EXAMPLE 1

The following composition, which is in cream form, is prepared.

The ingredients are collated in the table below, the amounts being expressed in grams of active material, unless otherwise indicated.

| | |
|---|---|
| Silica aerogel (Dow Corning VM-2270 Aerogel Fine Particles | 0.5 |
| sold by the company Dow Corning) | |
| Jojoba butter (Simmondsia chinensis (jojoba) butter; | 13 |
| iso jojoba 50 sold by the company Desert Whale) | |
| Hydrogenated polyisobutene (Parleam sold by the | 13 |
| company NOF) | |
| Synthetic wax (Cirebelle 303 sold by the company Cirebelle) | 5 |
| Cyclohexasiloxane | 13 |
| Dimethicone (and) dimethicone crosspolymer (Dow | 55.5* |
| Corning 9041 Silicone Elastomer Blend from Dow Corning) | |

*amount of commercial product

All of the starting materials except for the silica aerogel are placed in a heating pan, heated to 95° C. and homogenized using a Rayneri blender (300-400 rpm).

The silica aerogel is then added and the mixture is homogenized (500-700 rpm).

The composition is cooled in a jar to room temperature.

A creamy composition is then obtained.

When it is applied to bare lips, the appearance of the lips is considerably smoothed. The deposit is virtually invisible.

The application of a coat of standard lipstick (such as Color Riche from L′Oreal or Absolu from Lancôme), after deposition of the composition according to the invention, does not significantly change the makeup result thereof (colour, gloss, uniformity of the film) immediately after application.

On the other hand, after 2 hours, the contours of the lips are considerably sharper when the composition according to the invention is applied prior to the actual makeup composition.

In addition, the smoothing effect is still perceptible, even after application of the makeup.

Similar results are obtained with compositions prepared by replacing jojoba butter by the same amount of vinylpyrrolidone eicosene copolymer (Antaron V-220F by ISP) or hydrogenated cocoglycerides (Softisan 100 by Cremer Oleo).

### EXAMPLE 2

The ingredients are collated in the table below, the amounts being expressed in grams of active material, unless otherwise indicated.

| | |
|---|---|
| Talc | 1.5 |
| Silica aerogel (Dow Corning VM-2270 Aerogel Fine Particles | 0.5 |
| sold by the company Dow Corning) | |
| Synthetic wax (Cirebelle 108 sold by the company | 12 |
| Cirebelle) | |
| Jojoba butter (Simmondsia chinensis (jojoba) butter; | 18 |
| iso jojoba 50 sold by the company Desert Whale) | |
| Cyclohexasiloxane | 22 |
| Dimethicone (and) dimethicone crosspolymer (Dow Corning | 46* |
| 9041 Silicone Elastomer Blend from Dow Corning) | |

*amount of commercial product

All of the starting materials except for the silica aerogel and the talc are placed in a heating pan, heated to 95° C. and homogenized using a Rayneri blender (300-400 rpm).

The silica aerogel and the talc are then added and the mixture is homogenized, again with a Rayneri blender (500-700 rpm).

The resulting composition is then poured into a mould at 45° C.

When it is applied to bare lips, the appearance of the lips is considerably smoothed. The deposit is virtually invisible.

The application of a coat of standard lipstick (such as Color Riche from L'Oréal or Absolu from Lancôme), after deposition of the composition according to the invention, does not significantly change the makeup result thereof (colour, gloss, uniformity of the film) immediately after application.

On the other hand, after 2 hours, the contours of the lips are considerably sharper when the composition according to the invention is applied prior to the actual makeup composition.

In addition, the smoothing effect is still perceptible, even after application of the makeup.

1. A cosmetic composition, comprising:
at least 6% by weight, expressed as solids, relative to the weight of the composition, of at least one oraanopolysiloxane elastomer conveyed in a first oil,
at least one volatile or non-volatile non-phenyl silicone oil,
at least one apolar hydrocarbon-based compound, which is liquid or solid at room temperature, and
at least one pasty fatty substance.

2. The composition of claim 1, wherein the organopolysiloxane elastomer is conveyed in a first oil in the form of an organopolysiloxane elastomer gel.

3. The composition of claim 1, wherein the organopolysiloxane elastomer conveyed in the first oil non-emulsifying.

4. The composition of claim 1, wherein the organopolysiloxane elastomer(s) are present in a solids content ranging from 6% to 10% by weight, expressed as solids, relative to the total weight of the composition.

5. The composition of claim 1, wherein the non-phenyl silicone oil(s) is at least one of:
a non-volatile non-phenyl silicone oil selected from the group consisting of:
a polydimethylsioxane,
a polyalkylmethylsiloxane substituted with a functional group, and
a polysiloxane modified with a fatty acid, a fatty alcohol, a polvoxvalkylene, or mixtures thereof;
a volatile non-phenyl silicone oil which is a linear or cyclic silicone oils containing from 2 to 10 silicon atoms, optionally alkyl or alkoxy groups containing from 1 to 10 carbon atoms; and
mixtures thereof.

6. The composition of claim 1, wherein a content of the volatile or non-volatile, non-phenyl silicone oil(s) ranges from 25% to 95% by weight, to the weight of the composition.

7. The composition of claim 1, wherein the apolar hydrocarbon-based compound is a compound that is liquid at room temperature, and is selected from the group consisting of
a liquid paraffin or derivatives thereof,
squalane,
isoeicosane,
a naphthalene oil,
a hydrogenated or non-hydrogenated polybutene of polyisobutene,
a polydecene or a hydrogenated polydecene,
a decene/butene copolymer or a polybutene/polyisobutene copolymer, and
and mixtures thereof.

8. The composition of claim 1, wherein the apolar hydrocarbon-based compound is an apolar hydrocarbon-based compound that is solid at room temperature, and is selected from the group consisting of a microcrystalline wax, a microwax, a paraffin wax, an ozokerite, a polyethylene wax, a polymethylene wax and mixtures thereof.

9. The composition of claim 1, wherein a content of apolar hydrocarbon-based compound, which is liquid or solid at room temperature, ranges from 2% to 50% by weight, relative to the weight of the composition.

10. The composition of claim 1, wherein the fatty substance that is pasty at room temperature is a hydrocarbon based pasty fatty substance.

11. The composition of claim 1, wherein a content of pasty fatty substance is from 0.1% to 25% by weight relative to the weight of the composition.

12. The composition of claim 1, further comprising at least one filler in the form of fumed silica particles that have optionally been hydrophilic- or hydrophobic-treated.

13. The composition of claim 1, further comprising a content of silica or hydrophobic silica aerogels, expressed as active material, ranging from 0.1% to 10% by weight relative to the total weight of the composition.

14. A cosmetic process for making up and/or caring for the lips, the process comprising applying the composition of claim 1 to the lips.

15. The process of claim 14, wherein the composition is applied directly to the lips, and then optionally left to dry, before applying another composition, which is pigmented or unpigmented, and liquid or solid.

16. A method for preventing or limiting migration of a deposit resulting from application to the lips of a pigmented or unpigmented, liquid or solid composition, the method comprising applying the composition of claim 1 directly to the lips before applying the pigmented or unpigmented, liquid or sold composition.

17. A method for giving lips a smooth appearance, the method comprising applying the composition of claim 1 to the lips.
